(19) United States
(12) Patent Application Publication
(10) Pub. No.: US 2005/0143667 A1
(43) Pub. Date: Jun. 30, 2005

(54) Wireless heart rate sensing system and method

(76) Inventors: Jung-Min Park, Seoul (KR); Young-Bae Kwon, Incheon-city (KR); Seong-Ook Park, Daejeon-city (KR)

Correspondence Address: GIFFORD, KRASS, GROH, SPRINKLE & CITKOWSKI, P.C, PO BOX 7021, TROY, MI, 48007-7021, US

(21) Appl. No.: 10/865,706

(22) Filed: Jun. 10, 2004

(30) Foreign Application Priority Data
Dec. 26, 2003 (KR) 10-2003-0097581

(51) Int. Cl. A61B 5/0402

(52) U.S. Cl. 600/509

(57) ABSTRACT

Disclosed is a wireless heart rate sensing system which comprises: an oscillator for generating signals of a specific frequency; a power divider for dividing power of the signals generated by the oscillator; a transmit antenna for radiating a first signal output by the power divider to a patient's chest; a receive antenna for receiving a signal, the frequency of which is transited by a motion of the patient's chest and which is reflected and returned; a mixer for combining frequency components of an RF signal received through the receive antenna and a second signal output by the power divider; and a baseband unit for filtering the signal combined by the mixer, converting it into a digital signal, and outputting the digital signal.

### CROSS REFERENCE TO RELATED APPLICATION

This application claims priority to and the benefit of Korea Patent Application No. 10-2003-97581 filed on Dec. 26, 2003 in the Korean Intellectual Property Office, the entire content of which is incorporated herein by reference.

### BACKGROUND OF THE INVENTION

(a) Field of the Invention

The present invention relates to a system for sensing heart rates. More specifically, the present invention relates to a wireless system for sensing heart rates in real-time using circular polarization.

(b) Description of the Related Art

Many doctors in hospital settings measure heart rates by attaching electrodes of an ECG (electrocardiograph) meter to a human body, but this method allows measuring the heart rates only when attaching the electrodes to the body, and additionally requires manpower to manage equipment. Also, patents may feel resistance since they know their heart rates are being measured during the measuring process.

U.S. Pat. No. 3,934,577 entitled “Fetal heart rate monitoring apparatus” discloses a conventional wireless measuring system for heart rates.

As shown in FIG. 1, the patent uses a Doppler radar method, and in detail, it only filters signals which carry Doppler converted heart rate information, performs signal processing, and counts the number of heart beats. Further, the patent fails to disclose an RF system part but only shows a baseband part which is a signal processor.

In addition, Korea Patent No. 204980 entitled “Electromagnetic wave surface current meter” discloses a conventional technique which relates to a measuring device using the Doppler radar principle.

As shown in FIG. 2, the patent relates to a device for measuring the surface flux of watercourses using electromagnetic waves, which uses structures such as bridges built in the watercourses to shoot electromagnetic waves to the surface of a fluid, calculates a frequency according to the Doppler effect through the reflected and received signals, and uses the principle of converting the surface flux of the watercourses from the frequency. The device uses the Doppler radar principle, and one antenna performs both transmitting and receiving functions.

As described, the conventional techniques apply the Doppler radar method without considering an increase of isolation or widening a dynamic range of the system in most cases, and fail to represent data in real-time since the prior art collects the measured data without further applying any skills to antennas.

Also, the conventional breath or heart rate measuring system using the Doppler radar does not consider how to widen the dynamic range (an operational range) of the system. For example, a conventional system including a single antenna and using a circulator gives isolation characteristics of about −20.3 dB in the frequency bandwidth of 10 GHz, and the corresponding dynamic range of the system is saturated without reduction of the intensity of the power reflected on the patient's chest when the distance between the system and the patient is about 2.5 m. That is, the measured results by the conventional system cannot be relied when the operational range exceeds 2.5 m.

Further, the heart rates can be measured by a wired ECG meter which is generally used in hospitals and is used for measuring the patients' heart rates only when the electrode terminals are attached to the patient's body. Therefore, it is impossible to measure the patient when he is wounded or burned at a skin area on which the electrode terminals are to be attached.

Hence, it required for both a person to measure and a person to be measured to have a technique for representing wireless measuring of current heart rates of a patient in real-time.

### SUMMARY OF THE INVENTION

It is an advantage of the present invention to provide a system for measuring heart rates wirelessly and representing them in real-time.

It is another advantage of the present invention to provide a heart rate measuring system with compactness of size and convenience of manipulation.

In one aspect of the present invention, a wireless heart rate sensing system comprises: an oscillator for generating signals of a specific frequency; a power divider for dividing power of the signals generated by the oscillator; a transmit antenna for radiating a first signal output by the power divider to a patient's chest; a receive antenna for receiving a signal, the frequency of which is transited by a motion of the patient's chest and which is reflected and returned; a mixer for combining frequency components of an RF signal received through the receive antenna and a second signal output by the power divider; and a baseband unit for filtering the signal combined by the mixer, converting it into a digital signal, and outputting the digital signal.

The wireless heart rate sensing system further comprises a low noise amplifier, connected between the receive antenna and the mixer, for eliminating noise from the signal received through the receive antenna, maximizing a gain, and outputting a result signal.

The transmit antenna and the receive antenna are respectively a circular polarized antenna with a different polarized component.

The transmit antenna and the receive antenna have a patch antenna format in which a cavity is provided between the two antennas on a single substrate.

The baseband unit filters the signals through the IIR (infinite impulse response) filtering method.

In another aspect of the present invention, a wireless heart rate sensing method comprises: (a) power-dividing an oscillated signal and radiating it to a patient's chest; (b) receiving the signal, the frequency of which is transited by a motion of the patient's chest, and which is then reflected and returned; (c) combining frequency components of the received signal and the power-divided signal; and (d) filtering the combined signals, converting them into digital signals, and displaying them on a monitor.

### BRIEF DESCRIPTION OF THE DRAWINGS

The accompanying drawings, which are incorporated in and constitute a part of the specification, illustrate an embodiment of the invention, and, together with the description, serve to explain the principles of the invention:

FIG. 1 shows a conventional heart rate measuring device;

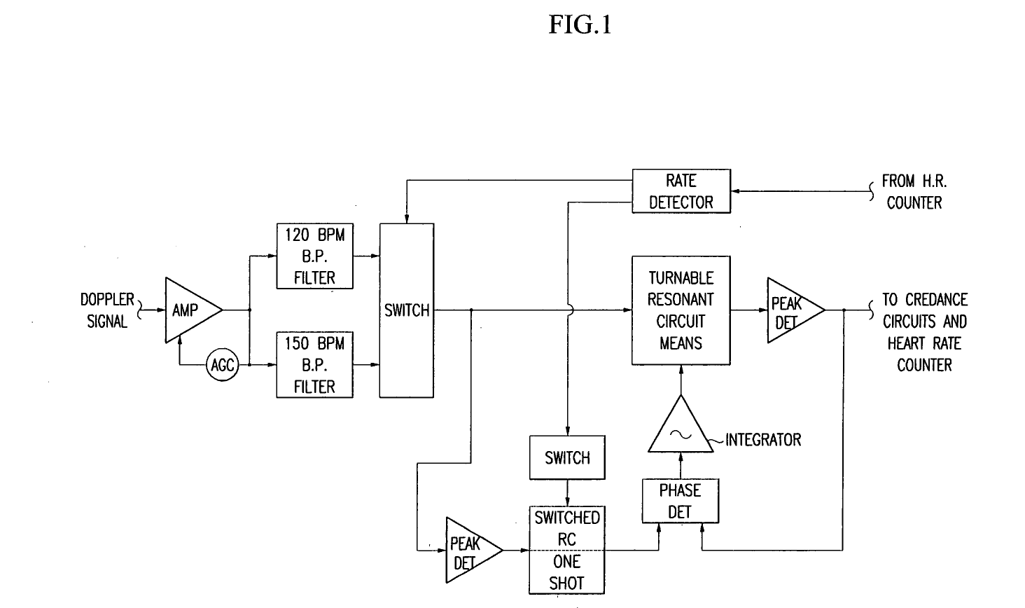

FIG. 2 shows a conventional measuring device using electromagnetic waves;

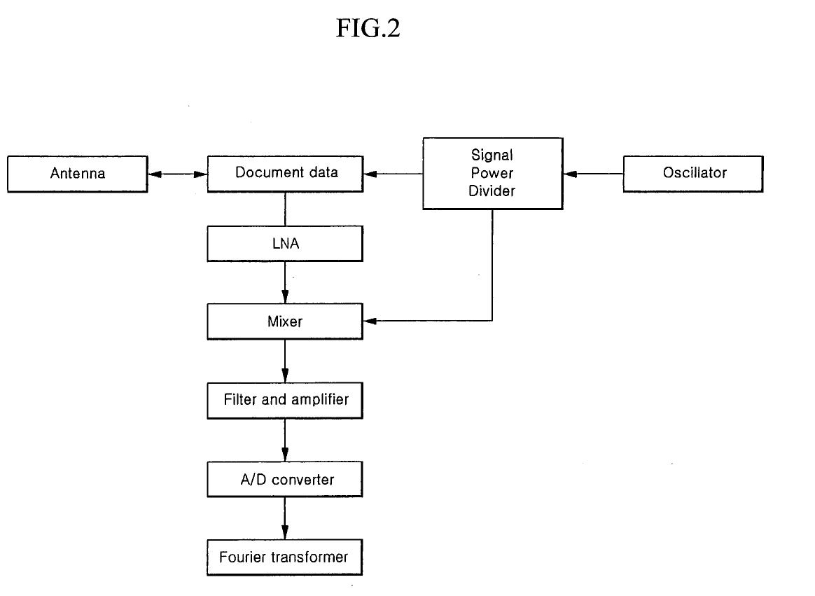

FIG. 3 shows a schematic diagram of a whole system according to a preferred embodiment of the present invention;

FIG. 4 shows a diagram comparing peaks of waveforms between the data collected by using the system according to a embodiment and a commercial ECG meter;

FIG. 5 shows a graph for comparing isolation measured results of a transmit antenna and a receive antenna and a circulator used for the heart rate sensing system according to a preferred embodiment of the present invention; and

FIG. 6 shows a configuration of a transmit antenna and a receive antenna for the heart rate sensing system according to a preferred embodiment of the present invention.

### DETAILED DESCRIPTION OF THE PREFERRED EMBODIMENTS

In the following detailed description, only the preferred embodiment of the invention has been shown and described, simply by way of illustration of the best mode contemplated by the inventor(s) of carrying out the invention. As will be realized, the invention is capable of modification in various obvious respects, all without departing from the invention. Accordingly, the drawings and description are to be regarded as illustrative in nature, and not restrictive. To clarify the present invention, parts which are not described in the specification are omitted, and parts which have similar descriptions have the same reference numerals.

A transmit antenna and a receive antenna having two different polarized components using circular polarization are used in the preferred embodiment to increase an isolation effect compared to the conventional method for performing both transmission and receiving functions with a single antenna, thereby widening the system's dynamic range, eliminating the noise component, and representing the heart rates by realizing a real-time filter in a software manner.

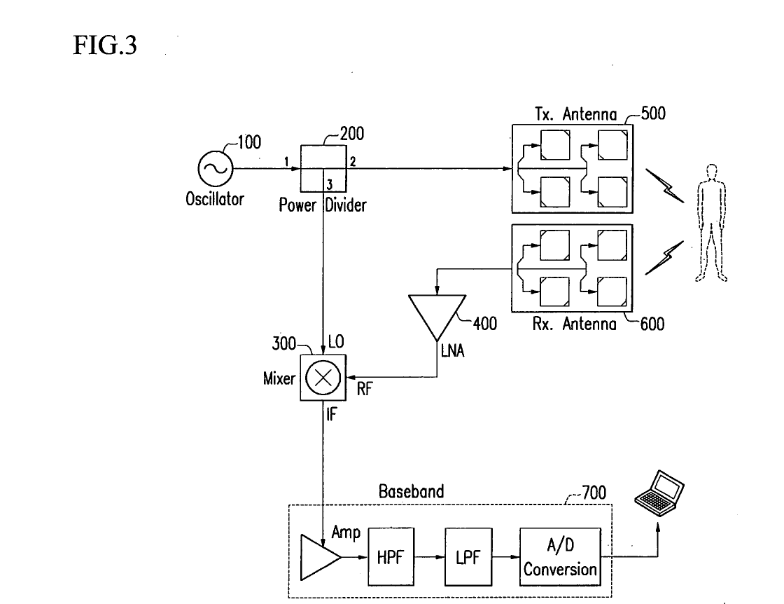

FIG. 3 shows a schematic diagram of a heart rate sensing system according to a preferred embodiment of the present invention.

As shown, the heart rate sensing system comprises a dielectric oscillator 100, a power divider 200, a mixer 300, an LNA (low noise amplifier) 400, a transmit antenna 500, a receive antenna 600, and a baseband unit 700.

The dielectric oscillator 100 outputs oscillation signals with the frequency of f₀.

The power divider 200 divides the signals output by the dielectric oscillator 100 into two signals and respectively transmits them to the mixer 300 and the transmit antenna 500.

The transmit antenna 500 radiates the input signal to the patient's chest.

The receive antenna 600 receives the signal which has been radiated to the patient's chest, the frequency of which has been transited and which has been reflected and returned from the patient, and outputs it to the LNA 400.

The LNA 400 maximally eliminates noise from the signal provided by the receive antenna 600, maximizes it gain, and transmits it to an RF (radio frequency) terminal of the mixer 300.

The mixer 300 mixes a signal input to an LO (local oscillator) terminal from the power divider 200 and the signal input to the RF terminal from the LNA 400 to output an IF (intermediate frequency) signal.

The baseband unit 700 processes the IF signal input from the mixer 300, converts it to a digital signal, and displays it on a monitor.

Next, an operation of the heart rate sensing system will be described in detail.

First, the signal with the frequency of f₀ oscillated by the dielectric oscillator 100 is divided into two signals through the power divider 200. One of the signals is input to the LO terminal of the mixer 300, and another one thereof is radiated to the patient's chest through the transmit antenna 500.

The signal radiated to the patient's chest from the transmit antenna 500 is frequency-transited by minute motion of the chest's outer skin such as the patient's heart beating and breathing, and is returned with the frequency of f₀±f₁.

The signal is then received by the receive antenna 600, noise thereof is eliminated by the LNA 400, and it is amplified and input to the RF terminal of the mixer 300.

The mixer 300 mixes the signals (the LO signal and the RF signal) having the frequencies of two components, and outputs signals which have information on the heart rates and breathing through an IF port of the mixer 300.

The analog signals output from the IF terminal of the mixer 300 are converted into digital signals, are filtered by the IIR (infinite impulse response) filtering method, and are then displayed on the monitor in real-time.

The transmit antenna 500 and the receive antenna 600 of the heart rate sensing system uses a patch antenna form using circular polarization, and transmits and receives signals with different polarized components.

The principle of the transmit antenna 500 and the receive antenna 600 will now be described.

The conductivity of the human skin and muscles is substantially 40 S/m in the frequency of 10 GHz, which is a very great value and is near that of a conductor. When a left-polarized signal is radiated on the conductor, it is converted to a right-polarized signal, and is returned. However, the left-polarized antenna transmits and receives the left-polarized signals, and the right-polarized antenna transmits and receives the right-polarized signals.

Therefore, the heart rate sensing system transmits the signals by using the left-polarized antenna, and receives the right-polarized signals reflected and returned from the skin near the heart through the right-polarized antenna. Through the process, undesired reflected signals are nor received but are eliminated, the isolation effect is improved, and the system's operational range is widened.

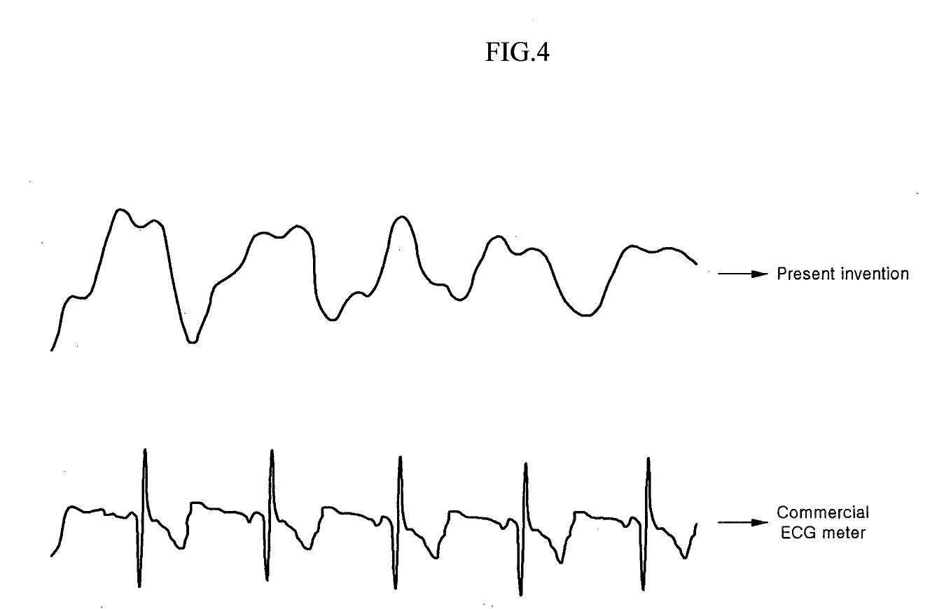

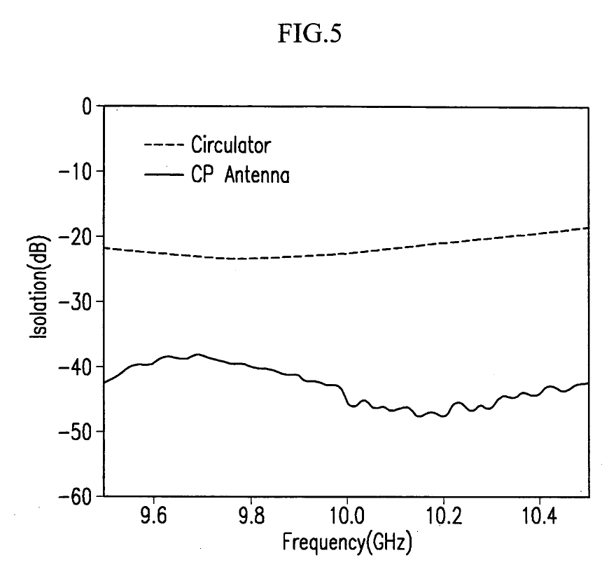

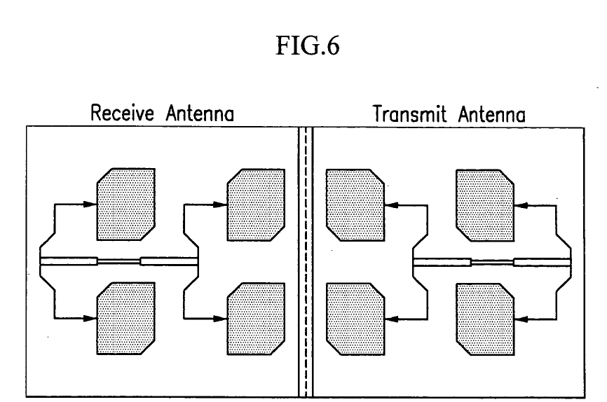

FIG. 4 shows a diagram comparing peaks of data waveforms between those measured by using the heart rate sensing system according to the embodiment and those measured by a commercial ECG meter; FIG. 5 shows a graph for comparing isolation measured results of the transmit antenna, the receive antenna, and a circulator used for the heart rate sensing system according to the preferred embodiment of the present invention; and FIG. 6 shows a configuration of the transmit antenna and the receive antenna for the heart rate sensing system according to the preferred embodiment of the present invention.

As shown, the heart rate sensing system obtains the isolation effect of about −45.2 dB near the frequency of 10 GHz through the transmit antenna and the receive antenna.

Also, the dimensions of the heart rate sensing system can be minimized by realizing an RF circuit operable in the frequency of 10 GHz and manufacturing a cavity between the transmit antenna and the receive antenna on a single substrate in a patch format as shown in FIG. 6, and the circulator can be eliminated from the heart rate sensing system by individually using the transmit antenna and the receive antenna.

While this invention has been described in connection with what is presently considered to be the most practical and preferred embodiment, it is to be understood that the invention is not limited to the disclosed embodiments, but, on the contrary, is intended to cover various modifications and equivalent arrangements included within the spirit and scope of the appended claims.

For example, the dielectric oscillator is exemplified for the oscillator in the preferred embodiment, and in addition, other types of various oscillators can be used.

As described, the heart rate sensing system is smaller and provides easy operation compared to the conventional ECG meter, and it accurately measures the patient's heart rate states without direct physical connection. Also, the heart rate sensing system has a wider operational range because of the high isolation effect.

1. A wireless heart rate sensing system comprising:
an oscillator for generating signals of a specific frequency;
a power divider for dividing power of the signals generated by the oscillator;
a transmit antenna for radiating a first signal output by the power divider to a patient's chest;
a receive antenna for receiving a signal, the frequency of which is transited by a motion of the patient's chest and which is reflected and returned;
a mixer for combining frequency components of an RF signal received through the receive antenna and a second signal output by the power divider; and
a baseband unit for filtering the signal combined by the mixer, converting it into a digital signal, and outputting the digital signal.

2. The wireless heart rate sensing system of claim 1, further comprising a low noise amplifier, connected between the receive antenna and the mixer, for eliminating noise from the signal received through the receive antenna, maximizing a gain, and outputting a result signal.

3. The wireless heart rate sensing system of claim 1, wherein the transmit antenna and the receive antenna are respectively a circular polarized antenna with a different polarized component.

4. The wireless heart rate sensing system of claim 1, wherein the transmit antenna and the receive antenna have a patch antenna format in which a cavity is provided between the two antennas on a single substrate.

5. The wireless heart rate sensing system of claim 1, wherein the baseband unit filters the signals through the IIR (infinite impulse response) filtering method.

6. A wireless heart rate sensing method comprising:
(a) power-dividing an oscillated signal and radiating it to a patient's chest;
(b) receiving the signal, the frequency of which is transited by a motion of the patient's chest, and which is then reflected and returned;
(c) combining frequency components of the received signal and the power-divided signal; and
(d) filtering the combined signals, converting them into digital signals, and displaying them on a monitor.

7. The wireless heart rate sensing method of claim 6, further comprising, between (b) and (c), eliminating noise from the received signals, and maximizing a gain.

8. The wireless heart rate sensing method of claim 7, wherein (a) and (b) comprise using a circular polarization phenomena with different polarized components, and transmitting and receiving signals.

9. A wireless heart rate sensing method comprising:
(a) power-dividing an oscillated signal, and radiating the signal to a patient's chest through an antenna with a circular polarized component in a first direction;
(b) receiving the signal, the frequency of which is transited by a motion of the patient's chest, and which is then reflected and returned, at an antenna with a circular polarized component in a second direction;
(c) combining frequency components of the received signal and the power-divided signal; and
(d) filtering the combined signals, converting them into digital signals, and displaying the digital signals on a monitor.

10. The wireless heart rate sensing method of claim 9, wherein the first direction is the right (or left) direction and the second direction is the left (or right) direction.
